(19) United States
(12) Patent Application Publication
(10) Pub. No.: US 2003/0143195 A1
(43) Pub. Date: Jul. 31, 2003

(54) Use of histamine as a drug delivery enhancing compound for use in transmucosal or transdermal delivery

(76) Inventor: Judy Senior Pinsker, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/066,513

(22) Filed: Jan. 30, 2002

(51) Int. Cl. A61K 39/00; A61K 38/21; A61K 38/20; A61K 31/4172

(52) U.S. Cl. 424/85.2; 424/85.5; 424/85.6; 514/400; 424/184.1

(57) ABSTRACT

A transmucosally administerable composition with enhanced penetration comprising: about 0.001% to about 2.5% of a delivery agent selected from the group consisting of histamine, histamine dihydrochloride, histamine phosphate, a pharmaceutically acceptable salt thereof, other histamine-receptor agonists, about 0.2% to about 75% of a pharmaceutically active medicament, about 0% to about 99.8% of solvent, and about 0% to about 15% of a gelling agent.

### RELATED APPLICATIONS

This application claims the benefit of priority from PCT/US00/20757, filed Jul. 28, 2000, published under PCT Article 21(2) in English and which claims the benefit of priority from Provisional Application No. 60/146,641, each of which is hereby incorporated by reference in their entirety.

### BACKGROUND OF THE INVENTION

1. Field of the Invention

The disclosed invention relates to compositions and methods for enhancing delivery of a pharmaceutical or therapeutic agent to a subject. Specifically, the disclosed invention includes methods and compositions for augmented transmucosal delivery of drugs and vaccines.

2. Description of the Related Art

Various types of drug delivery systems are well known in the prior art. Possibly the most common of these systems is an intravenous drip system for the delivery of drugs to bedridden patients. In one embodiment of this system, an elevated container with a valve controlling the drip rate of the drug into a tube is coupled with a needle inserted into the patient's body. With such a system, the flow rate may be controlled by means of a valve. This system presents a number of problems, not the least of which is its limitation for use only with non-ambulatory patients. Similarly, drugs may be delivered intravenously by operation of a low volume pump. However, most systems employing pumps are rather large and require a reliable source of power for proper operation. In addition, these devices are typically limited to use with bedridden patients.

Delivery of drugs into a body cavity is typically accomplished systemically. Systemic drug delivery through oral, intravenous, or intramuscular administration methods carries with it the obvious drawbacks of any systemic treatment, such as side effects. The drug can also be metabolized or altered by physiological processes, and the ultimate quantity of active drug that reaches the body cavity is be reduced. In addition, because many drugs are not well tolerated systemically, the dosage must be limited, thereby reducing the total effective dose that reaches the targeted body cavity.

Numerous attempts have been made to optimize drug delivery. One approach is to deliver drugs transdermally. Transdermal administration systems are well known in the art. See, e.g. U.S. Pat. Nos. 4,839,174, 4,908,213 and 4,943,435. Transmucosal administration of drugs is yet another approach to optimizing drug delivery. Transmucosal delivery of drugs avoids first-pass inactivation, inactivation by gastrointestinal fluids, and other modes of inactivation characteristic of oral drug ingestion.

While transmucosal delivery of drugs overcomes many of the drawbacks associated with traditional drug delivery mechanisms, there remains a need for improved methods and compositions for optimized drug delivery.

### SUMMARY OF THE INVENTION

The disclosed invention is directed to methods and compositions for enhancing transmucosal or transdermal delivery of a drug or vaccine. One embodiment of the disclosed invention is a transmucosally administrable composition with enhanced penetration. Advantageously, the composition includes between about 0.001% to about 25% of a permeation enhancing agent, wherein the permeation enhancing agent includes histamine, histamine dihydrochloride, histamine phosphate, a pharmaceutically acceptable salt thereof, or other histamine agonists. In one embodiment of the invention, the composition additionally includes from about 0.2% to about 90% of a therapeutically active medicament, about 0% to about 99.8% of a solvent; and from about 0% to about 50% of a gelling agent. Optionally, the composition includes an absorption enhancer. The absorption enhancer may include sulphoxides, alcohols, polyols, alkanes, fatty acids, esters, amines, amides, terpenes, surfactants, cyclodextrins, dimethylsulphoxide, pyrrolidones, N,N-diethyl-m-toluamide, or laurocapram.

In another aspect of the invention, a transmucosally administrable composition is provided. The composition may advantageously include a therapeutic compound and a permeation enhancing agent. Preferably, the permeation enhancing agent is an effective amount of histamine dihydrochloride, histamine phosphate, or related salts.

The contemplated pharmaceutically active medicament can include IL-2, IL-12, IL-15, IFN-α, IFN-β, antivirals, analgesics, pain relievers, antibiotics, peptides, proteins, vitamins, other chemotherapeutic agents, vaccines, or any other pharmaceutically active compound that can be efficaciously administered through transmucosal membranes. Advantageously, the pharmaceutically active medicament may include mixtures of the above-enumerated therapeutic compounds.

The disclosed invention further contemplates a method of administering a pharmaceutically active compound to the buccal mucosa including contacting a mucosal membrane with a transmucosally administrable composition having enhanced penetration. Preferably, the transmucosally administrable composition with enhanced penetration includes about 0.001% to about 25% of a permeation enhancing agent wherein the permeation enhancing agent is histamine, histamine dihydrochloride, histamine phosphate, a pharmaceutically acceptable salt thereof, or other histamine agonists alone or in combination with other enhancing agents. Advantageously, the composition may additionally include about 0.2% to about 90% of a therapeutically active medicament, about 0% to about 99.8% of solvent; and about 0% to about 50% of a gelling agent.

A method of manufacture of a pharmaceutical composition for administration to the buccal mucosa is likewise provided. The method includes providing a therapeutic compound and a permeation enhancing agent. The permeation enhancing agent includes histamine, histamine dihydrochloride, histamine phosphate, histamine agonists, or histamine salts. Advantageously, the therapeutic compound and permeation enhancing agent are in a pharmaceutically acceptable form. Optionally, the method of manufacture includes incorporating the pharmaceutical composition into a transmucosal delivery system.

### DETAILED DESCRIPTION OF THE PREFERRED EMBODIMENT

The disclosed invention relates to compositions for transmucosal delivery of various compounds, e.g., for use in transmucosal or transdermal administration of drugs and vaccines. Additionally, a method of augmenting the transmucosal delivery of various compounds is likewise contemplated.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

It will be appreciated that the term “transmucosal” refers to delivery of a drug through the mucosa or skin and thus will include a transdermal route of drug administration. As used herein, “drug,” “pharmaceutical agent,” “pharmacological compound,” “therapeutic agent” or any other similar term means any chemical or biological material or compound suitable for transmucosal and/or transdermal administration by methods previously known in the art and/or by the methods and compositions taught in the disclosed invention that induce a desired biological or pharmacological effect, which can include but is not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating the disease from the organism. The effect can be local, such as providing for a local anesthetic effect, or it can be systemic.

Transmucosal administration of drugs offers advantages over other routes of drug administration. For example, drugs administered through the buccal and sublingual membrane routes have a rapid onset of action, reach high levels in the blood, avoid the first-pass effect of hepatic metabolism, and avoid exposure of the drug to fluids of the gastrointestinal tract. Additional advantages include easy access to the membrane sites so that the drug can be administered, localized, and removed easily. Further, there is potential for prolonged delivery through the buccal membrane. M. Rathbone & J. Hadgraft, 74 Int'l J. of Pharmaceutics 9 (1991). Mucosa is relatively permeable, thus providing rapid absorption and acceptable bioavailabilities of many drugs. This route has been investigated clinically for the delivery of a substantial number of drugs.

Administration of pharmaceutical compounds using a transmucosal or transdermal route of administration provides a number of advantages over conventional injection modalities. One advantage over a traditional injection method is that transmucosal administration does not involve the use of a hypodermic needle and the attendant risks to achieve local and systemic compound administration. An example of non-injection drug administration is transdermal administration.

Although transdermal administration is efficient, drug absorption may be enhanced and improved when applied to a mucosal surface instead of the transdermal route of administration. Transmucosal administration of pharmaceutical compounds is advantageous given a number of physiological characteristics of the mucosa itself. For example, mucosal surfaces are usually rich in blood supply, providing the means for rapid drug transport to the systemic circulation and avoiding, in most cases, degradation by first-pass hepatic metabolism. Similarly, the increased efficiency of drug absorption when applied transmucosally can be attributed to the absence of the stratum corneum epidermidis. By contrast, with transdermal administration of drugs, the stratum corneum epidermidis acts as the major barrier to absorption across the skin.

A variety of factors determine the rate and amount of pharmaceutical compound absorbed through the mucosa. These factors include the concentration of the pharmaceutical compound applied to the mucosa, the vehicle of drug delivery used, the duration of mucosal contact, the amount of venous drainage of the mucosal tissue to which the compound(s) is applied, the chemical state of the pharmaceutical composition including the extent of ionization of the composition, the pH of the absorption site, the molecular weight of the various components in the pharmaceutical composition, the hydrophobicity of those components, and the presence of an uptake accelerant or mucosal membrane delivery enhancing agent.

In one embodiment, the disclosed invention is directed to enhancing drug administration via transmucosal delivery of a pharmaceutical composition with a permeation enhancer. As used herein, the term “permeation enhancer” or “permeation enhancing agent” includes substances that facilitate the transport of solutes across biological membranes. Such substances include histamine and histamine related compounds, including histamine, histamine dihydrochloride, histamine diphosphate, other histamine salts, and histamine agonists. Similarly, it will be appreciated that the term “histamine”, as used herein, includes histamine and all histamine related compounds described herein.

Advantageously, permeation enhancers can be used as uptake accelerants or mucosal permeation enhancing agents in transmucosally administered compositions. While not intending to be limited to any particular theory, it is thought that these compounds function as mucosal permeation enhancing agents by stimulating mucosal blood flow and increasing capillary permeability. These circulatory changes caused by histamine to a site upon which an effective amount of a drug or vaccine of interest is applied result in an increase in the uptake of that drug or vaccine. Thus, histamine may facilitate delivery of pharmaceutical compounds via transdermal and transmucosal routes.

The delivery-enhancing component is a substance which functions to assist in the migration of the pharmaceutically active component(s) through the membranes and into the bloodstream. Thus, any mucosal membrane can be a target site for administration. Specific examples of suitable mucosal membranes upon which the compositions can be applied include: nasal, ophthalmic, oral, intestinal, rectal, vaginal, and penile membranes.

The transmucosal permeation enhancing agents described herein enhance the transmucosal delivery of various compounds of interest. Suitable permeation enhancing agents include histamine, its various pharmaceutically acceptable salt forms such as histamine dihydrochloride, histamine phosphate, other histamine agonists, and the like. Advantageously, the compositions of the disclosed invention contains between about 0.001% to about 25% by weight of the permeation enhancing agent. All percentages recited herein are weight percentages based on total composition weight unless otherwise specified. In preferred embodiments, an effective dose of the permeation enhancing agent is about 0.001%, 0.005%, 0.01%, 0.5%, 0.1%, 0.5%, 1.0%, 1.5%, 2%, 5%, 10%, 15%, 20%, or about 25% by weight of the formulation. Mixtures of histamine and histamine related compounds as permeation enhancing agents are also contemplated as falling within the scope of the disclosed invention.

In some embodiments, the composition includes a known absorption enhancer in a pharmaceutically acceptable form in addition to a permeation enhancing agent listed above. As used herein, the term “absorption enhancer” includes known substances which facilitate absorption of drugs through the skin or mucosa. Suitable absorption enhancers include sulphoxides, alcohols, polyols, alkanes, fatty acids, esters, amines, amides, terpenes, surfactants, cyclodextrins, dimethylsulphoxide, pyrrolidones, N,N-diethyl-m-toluamide, and laurocapram. The co-administration of histamine with an absorption enhancer further augments rapid drug delivery across the skin and mucosa membranes.

In addition to the permeation enhancing agent, the transmucosal delivery composition includes a pharmaceutically active compound of interest to be transferred across the mucosal membrane. A variety of therapeutic compounds such as various drugs and vaccines are contemplated for use with the transmucosal permeation enhancing agents described herein. Such therapeutic compounds include broad classes of compounds normally delivered into the body through body surfaces and membranes, including the mucosa and skin. In general these include but are not limited to: anti-infectives such as antibiotics and antiviral agents; analgesics and analgesic combinations; anorexics; antihelminthics; antiarthritics; antiasthmatic agents; anticonvulsants; antidepressants; antidiabetic agents; antidiarrheals; antiinflammatory agents; antimigraine preparations; antinauseants; antineoplastics; antiparkinsonism drugs; antipruritics; antipsychotics; antipyretics; antispasmodics; anticholinergics; syphathomimetics; xanthine blockers, alpha-blockers, antiarrhythmics; antihypertensives; diuretics and antidiuretics; vasodilators including general coronary, peripheral, and cerebral; central nervous system stimulants; nutroceuticals, vasoconstrictors; cough and cold preparations, including decongestants; hormones such as estradiol and other steroids, hypnotics; immunosuppressives; muscle relaxants, parasympatholytics; psychostimulants; peptides; proteins; protease inhibitors, vitamins; cytokines such as IL-2, IL-12, IL-15, IFN-α, and IFN-β; other chemotherapeutic agents; sedatives; tranquilizers; and any other pharmaceutically active compound that can be efficaciously administered through a transmucosal membrane. One of skill in the art will appreciate that the pharmaceutically active compounds listed above would be formulated in a pharmaceutically acceptable and substantially non-toxic amount according to procedures well known in the art.

In some embodiments, the therapeutic compound for transmucosal delivery is a vaccine. The vaccine may be directed against an organism such as a virus or bacterium to increase immunity to a particular disease. Alternatively, the vaccine may target neoplastic cells.

Generally, the pharmaceutically active component will comprise between about 0.1% to about 90% of the composition. Preferably, the compounds of interest are contained in a composition comprising a pharmaceutically active compound in an amount of between about 0.1% to about 50% by weight to volume of the total composition. In a particularly preferred embodiment, the pharmaceutically active component may advantageously comprise between about 0.2% to about 30% by weight to volume of the total composition. Cytokines may be administered in a daily dose to an adult human between about 1000 to about 600,000 U/kg.

Additional components of the transmucosal delivery compositions contemplated for use as described herein are well known in the art. Such vehicles include water; organic solvents such as alcohols (such as ethanol); glycols (such as propylene glycol); aliphatic alcohols (such as lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); hydrocarbon-based materials such as microsponges and polymer matrices; stabilizing and suspending agents; emulsifying agents; and other vehicle components that are suitable for administration to the skin, as well as mixtures of these components and those otherwise known in the art.

Advantageously, the transmucosal delivery compositions include components adapted to improve the stability or effectiveness of the applied formulation, such as preservatives, antioxidants, and sustained release materials. Examples of such components are described in the following reference works hereby incorporated by reference: Martindale—The Extra Pharmacopoeia (Pharmaceutical Press, London 1993) and Martin (ed.), Remington's Pharmaceutical Sciences.

Solvents, waxes, emulsifying agents, stabilizers, preservatives, antioxidants, sustained release materials, and other vehicles described above can be included in the composition in varying amounts. In one embodiment, the composition includes between about 0% to about 99.8% by weight of solvent. Advantageously, the composition additionally comprises between about 0% to about 50% by weight of a gelling agent and other excipients or diluents as would occur to one skilled in the art. All acceptable excipients, antioxidants, preservatives, including WICKENOL 535 (Wickhen Product Inc., a mixture of mono-, di-, and tri-glyceride of wheat germ oil) and Vitamin E are within the scope of the instant invention.

Specific embodiments of the disclosed invention include a transmucosally administered composition comprising: about 0.001%, 0.005%, 0.01%, 0.05%, 0.1%, 0.5%, 1.0%, 5%, 10%, 15%, 20% or about 25% histamine dihydrochloride, and between about 0.5% to about 90% of a pharmaceutically active medicament by weight of the total composition. Preferably, the pharmaceutically active medicament is added in an amount of between 0.5% to 50% by weight of the composition. More preferably, the pharmaceutically active medicament is added in an amount of between 0.5% and 25% by weight of the composition.

In additional embodiments, a suitable solvent, such as water, oil, ethanol, butylene glycol, propylene glycol, isopropyl alcohol, isoprene glycol, glycerine, isopropylmyristate, and the like is present in the composition in an amount from between about 0.1% to about 99.8% by weight of the formulation. Preferably, a solvent is added to the formulation in an amount ranging from about 50% to about 95% by weight. More preferably, a solvent is added to the formulation from between about 75% to about 90% by weight. In addition, combinations or mixtures of these solvents can be used in the formulations described herein. The term solvent is intended to include that portion of the formulation which provides the flux of the active ingredient across the skin or mucosa.

In a preferred embodiment, the formulation further comprises between about 0% to about 25% of a suitable gelling agent such as AEROSIL 200 (B.F. Goodrich, Cleveland, Ohio). Advantageously, the concentration of the gelling agent is from about 0.5% to about 15% by weight of the total formulation. In a particularly preferred embodiment, the gelling agent comprises between about 5% to about 10% by weight of the formulation.

The transmucosal compositions described herein can also contain preservatives including, but not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. In addition, combinations or mixtures of these preservatives can be used in the formulations disclosed herein.

A variety of delivery systems and devices are available to administer the transmucosal compositions to a subject. The drug delivery system is preferably embodied in either a device of determined physical form, such as a tablet, patch, or troche, or in free form, such as a gel, ointment, cream, or suppository. Similarly, the compositions can be administered by drops, solution sprays, suspension sprays, powders, emulsions, microspheres, liposomes, steady state reservoirs, and the like. Transdermal patches, steady state reservoirs sandwiched between an impervious backing and a membrane face, and transdermal formulations can also be used to transmucosally or transdermally deliver permeation enhancers such as histamine in concert with drugs or vaccines. One suitable type of transdermal patch is a polymer matrix in which the active agent is dissolved in a polymer matrix through which the active ingredient diffuses to the skin. Such transdermal patches are disclosed in U.S. Pat. Nos. 4,839,174, 4,908,213 and 4,943,435, the subject matter of which are hereby incorporated by reference in their entirety.

In a preferred embodiment, histamine, in combination with a pharmaceutically active compound, is released from a transdermal patch at a rate of between about 0.001 mg to 20 mg per minute for a dose of between about 0.01 mg to 150 mg histamine per patch. The use of electrolytic transdermal patches is also within the scope of the invention disclosed herein. Electrolytic transdermal patches are described in U.S. Pat. Nos. 5,474,527, 5,336,168, and 5,328,454.

Hydrogels, wherein the permeation enhancing agent such as histamine is dissolved in an aqueous constituent to gradually release over time, can be prepared by copolymerization of hydrophilic (mono-)olefinic monomers such as ethylene glycol methacrylate. Matrix devices, wherein the histamine or a histamine related compound is dispersed in a matrix of carrier material, can also be used to transmucosally deliver drugs. The carrier can be porous, non-porous, solid, semi-solid, permeable or impermeable. Alternatively, a device comprising a central reservoir of histamine surrounded by a rate controlling membrane can be used to control the release of histamine. Rate controlling membranes include ethylene-vinyl acetate copolymer or butylene terephthalate/polytetramethylene ether terephthalate. Use of silicon rubber depots are also contemplated.

In particularly preferred embodiments, transmucosal patches designed for placement over mucosal tissue are used to administer a pharmaceutical composition with histamine as a permeation enhancer. An example of such a patch is found in U.S. Pat. No. 5,122,127. The described patch comprises a housing capable of enclosing a quantity of therapeutic agent where the housing is capable of adhering to mucosal tissues, for example, in the mouth. A drug surface area of the device is present for contacting the mucosal tissues of the host. The device is designed to deliver the drug in proportion to the size of the drug/mucosa interface area. Accordingly, drug delivery rates may be adjusted by altering the size of the contact area.

The housing is preferably constructed of a material that is nontoxic, chemically stable, and non-reactive with the compounds disclosed herein. Suitable construction materials include: polyethylene, polyolefins, polyamides, polycarbonates, vinyl polymers, and other similar materials known in the art. The housing can contain means for maintaining the housing positioned against the mucosal membrane. The housing can contain a steady state reservoir positioned to be in fluid contact with mucosal tissue.

Steady state reservoirs for use with the compounds disclosed herein will deliver a suitable dose of those compounds over a predetermined period of time. Compositions and methods of manufacturing compositions capable of absorption through the mucosal tissues are taught in U.S. Pat. No. 5,288,497. One of skill in the art would readily know how to include the compounds of the invention disclosed herein in these and related compositions.

A method of administering the above-described composition includes combining histamine and a pharmaceutically active compound with a vehicle to produce a transmucosal composition, as described above. The resulting formulation is then incorporated into a suitable device for delivery to a body membrane and absorption therethrough.

A method of manufacturing a pharmaceutical composition for transdermal administration is similarly contemplated by the disclosed invention. Advantageously, a therapeutic compound in a pharmaceutically acceptable form is combined with a permeation enhancing agent to form a pharmaceutical composition. The permeation enhancing agent may include histamine, histamine dihydrochloride, histamine phosphate, histamine dihydrochloride, histamine agonists and other histamine salts in a pharmaceutically acceptable form. In a preferred embodiment, a solvent and/or gelling agent is likewise added to the composition. Advantageously, the composition is incorporated into a suitable drug delivery system for administration to the mucosal membranes. For example, the composition can be added to a transmucosal patch, cream, ointment, gel, spray, emulsion, ointment, suppository, and the like.

The following Examples are given by way of illustration only and are not intended to serve as a limitation to the scope of the disclosed invention. The Examples are provided to give a clear understanding of the disclosed invention and a manner in which the invention can be performed.

### EXAMPLE 1

A subject in need of insulin is provided a transmucosal patch comprising an effective dose of insulin and histamine dihydrochloride at 0.1% by weight of formulation. The transmucosal patch is described in U.S. Pat. No. 5,750,136, which is hereby incorporated by reference. The transmucosal patch containing the insulin and the histamine dihydrochloride is applied to the buccal mucosa, where it is held in fluid communication with the mucosa. The histamine dihydrochloride present in the patch is transferred by diffusion from the patch to the mucosa. Molecules of insulin also pass into the mucosa and then into the bloodstream of the subject wearing the transmucosal patch. The histamine dihydrochloride enhances the delivery of the insulin into the subject's bloodstream.

### EXAMPLE 2

A subject presenting with pain is treated with a gel comprising an effective dose of an analgesic and histamine phosphate in a concentration of 1.0% by weight of the formulation. The gel containing an analgesic and the histamine diphosphate is applied to the oral mucosa. The histamine phosphate in the gel is diffused from the gel to the mucosa and molecules of analgesic pass into the mucosa and into the bloodstream of the subject. The histamine phosphate enhances delivery of the analgesic into the subject's bloodstream.

### EXAMPLE 3

A female subject suffering from herpes genitalis is treated with a compound of the disclosed invention. The compound is prepared in a cream for transmucosal application according to procedures well known in the art. The permeation enhancing agent, a histamine agonist, in a concentration of 5% by weight of formulation is added to the cream. The cream additionally contains 9-(2-Hydroxyethoxymethyl)guanine, ZOVIRAX, (Glaxo Wellcome) in a pharmaceutically appropriate dosage. Using an applicator, the cream is injected into the vaginal space to treat herpetic lesions therein. The permeation enhancing agent increases the effectiveness of the ZOVIRAX.

### EXAMPLE 4

A patient presenting with HIV infection is administered a suppository composition comprising an effective dose of a protease inhibitor and histamine phosphate in a concentration of 5% by weight of the formulation. The suppository containing a protease inhibitor and the histamine phosphate is inserted rectally. The histamine phosphate in the suppository formulation is diffused from the suppository to the mucosa and molecules of the protease inhibitor pass into the mucosa and into the bloodstream of the subject. The histamine phosphate enhances the delivery of the protease inhibitor.

### EXAMPLE 5

A patient presenting with a vitamin deficiency, such as beriberi or scurvy, is nasally administered a powder comprising an effective dose of a vitamin and 0.1% histamine dihydrochloride by weight of formulation. The histamine dihydrochoride in the powder is diffused across the nasal mucous membrane and the vitamin passes into the mucosa and into the subject's bloodstream. The histamine dihydrochloride enhances the delivery of the vitamin into the subject's bloodstream.

### EXAMPLE 6

A subject presenting with neoplastic disease is administered an oral spray containing 300,000 U/kg IL-2 and 0.5% histamine phosphate by weight of the formulation. The oral spray containing IL-2 and histamine phosphate is prepared according to procedures well known in the art. The histamine phosphate is diffused from the pharmaceutical spray to the oral mucosa and molecules of IL-2 travel from the mucosa and into the bloodstream of the subject. The histamine phosphate enhances the delivery of IL-2 into the subject's bloodstream.

### EXAMPLE 7

A vaccine comprising attenuated chickenpox virus and 0.75% histamine by weight of the vaccine is formulated in a transmucosal patch. The patch is applied to the buccal mucosa of a subject. The histamine acts as a permeation enhancing agent. The attenuated chickenpox virus readily travels from the mucosa and into the bloodstream of the subject. An immune response is mounted by the subject against the chickenpox virus.

### CONCLUSION

The above-described invention relates the use of various permeation enhancing agents including histamine, to administer pharmaceutically active compounds of interest via a mucosal membrane.

Although the invention has been described with reference to embodiments and various examples, it should be understood that various modifications can be made without departing from the spirit of the invention. Accordingly, the invention is limited only by the following claims.

What is claimed is:

1. A transmucosally administrable composition comprising:
a pharmaceutically active compound; and
a permeation enhancing agent, wherein said agent is selected from the group consisting of histamine, histamine dihydrochloride, histamine phosphate, a pharmaceutically acceptable salt thereof, and other histamine agonists.

2. The transmucosally administrable composition of claim 1 wherein said composition comprises about 0.001% to about 25% weight/volume of said permeation enhancing agent.

3. The transmucosally administrable composition of claim 1, wherein said permeation enhancing agent is histamine dihydrochloride.

4. The transmucosally administrable composition of claim 1, wherein said permeation enhancing agent is histamine phosphate.

5. The transmucosally administrable composition of claim 1, wherein said permeation enhancing agent is histamine.

6. The transmucosally administrable composition of claim 1, wherein said composition comprises about 0.2% to about 90% of said pharmaceutically active compound.

7. The transmucosally administrable composition of claim 1, wherein said composition further comprises about 0% to about 99.8% of a solvent.

8. The transmucosally administrable composition of claim 1, wherein said composition further comprises about 0% to about 50% of a gelling agent.

9. The transmucosally administrable composition of claim 6, wherein the pharmaceutically active compound is a therapeutic compound selected from the group consisting of: IL-2, IL-12, IL-15, IFN-α, IFN-β, antivirals, analgesics, pain relievers, antibiotics, peptides, proteins, vitamins, other chemotherapeutic agents, vaccines, and any other pharmaceutically active compound that can be efficaciously administered through a transmucosal membrane, and mixtures thereof.

10. The transmucosally administrable composition of claim 1, further comprising an absorption enhancer in a pharmaceutically acceptable form.

11. The transmucosally administrable composition of claim 10, wherein said absorption enhancer is selected from the group consisting of sulphoxides, alcohols, polyols, alkanes, fatty acids, esters, amines, amides, terpenes, surfactants, cyclodextrins, dimethylsulphoxide, pyrrolidones, N,N-diethyl-m-toluamide, and laurocapram.

12. A method of administering a pharmaceutically active compound to the buccal mucosa comprising contacting a composition of claim 1 with a mucosal membrane.

13. The method of claim 12, further contacting the mucosal membrane with an absorption enhancer.

14. The method of claim 13, wherein said absorption enhancer is selected from the group consisting of sulphoxides, alcohols, polyols, alkanes, fatty acids, esters, amines, amides, terpenes, surfactants, cyclodextrins, dimethylsulphoxide, pyrrolidones, N,N-diethyl-m-toluamide, and laurocapram.

15. A method of manufacture of a pharmaceutical composition for administration to the buccal mucosa comprising:
providing a therapeutic compound and a permeation enhancing agent selected from the group consisting of histamine, histamine dihydrochloride, histamine phosphate, histamine agonists, and histamine salts in a pharmaceutically acceptable form; and
incorporating said therapeutic compound and said permeation enhancing agent into a transmucosal delivery system.

16. The method of manufacture of claim 15, further comprising incorporating into said transdermal delivery system an absorption enhancing agent.

17. The method of claim 16, wherein said absorption enhancing agent is selected from the group consisting of sulphoxides, alcohols, polyols, alkanes, fatty acids, esters, amines, amides, terpenes, surfactants, cyclodextrins, dimethylsulphoxide, pyrrolidones, N,N-diethyl-m-toluamide, and laurocapram.
